(19) United States
(12) Patent Application Publication
(10) Pub. No.: US 2026/0335021 A1
(43) Pub. Date: Oct. 29, 2026

(54) SURGICAL ASSISTANCE USING ARTIFICIAL INTELLIGENCE

(71) Applicant: ALCON INC., Fribourg (CH)

(72) Inventors: Sinchan Bhattacharya, Fort Worth, TX (US); Vignesh Suresh, Fort Worth, TX (US); Aakash Nagarajan, Arlington, TX (US); Ramesh Sarangapani, Coppell, TX (US); Nikhil Scaria, Fort Worth, TX (US); Zhuoran Wu, Rancho Cucamonga, CA (US); Lu Yin, Fort Worth, TX (US)

(21) Appl. No.: 19/645,085

(22) Filed: Apr. 10, 2026

Related U.S. Application Data
(60) Provisional application No. 63/794,946, filed on Apr. 25, 2025.

(51) Int. Cl. A61B 34/20; G06T 7/246; G06T 7/37; G06V 10/764

(52) U.S. Cl. CPC A61B 34/20; G06T 7/251; G06T 7/37; G06V 10/764; A61B 2034/2065; G06T 2207/10016; G06T 2207/30196

(57) ABSTRACT

A method of tracking an incision may include obtaining frames of video data captured during a medical procedure and providing the frames of the video data to an artificial-intelligence classifier model configured to detect when a medical tool initiates an incision in tissue. The method may also include in response to initiation of the incision, generating properties of the incision using one or more artificial-intelligence tracking models configured to analyze frames of the video data as the incision is made and determining, based on the properties of the incision, a location of the incision in a subsequent frame of the video data after the incision is completed.

### CROSS-REFERENCE TO RELATED APPLICATION

This application claims priority to U.S. Provisional Application No. 63/794,946, filed on Apr. 25, 2025, which is incorporated herein in its entirety.

### FIELD

The embodiments discussed herein relate to a surgical assistance using artificial intelligence.

### BACKGROUND

Surgical video analysis systems process video streams captured during medical procedures to extract information about surgical events, anatomical structures, and instrument positions. For example, modern surgical microscopes and endoscopes may generate high-resolution digital video that document a surgical procedure. Computer vision algorithms applied to the digital video may focus on detecting and tracking surgical instruments, identifying anatomical landmarks, or recognizing coarse-grained surgical phases that span multiple minutes. For example, existing systems may employ artificial intelligence (AI) models to locate and track surgical tools in the digital video.

The subject matter claimed herein is not limited to embodiments that solve any disadvantages or that operate only in environments such as those described above. Rather, this background is only provided to illustrate one example technology area where some embodiments described herein may be practiced.

### SUMMARY

According to an aspect of at least one embodiment, a method of tracking an incision may include obtaining frames of video data captured during a medical procedure and providing the frames of the video data to an artificial-intelligence classifier model configured to detect when a medical tool initiates an incision in tissue. The method may also include in response to initiation of the incision, generating properties of the incision using one or more artificial-intelligence tracking models configured to analyze frames of the video data as the incision is made and determining, based on the properties of the incision, a location of the incision in a subsequent frame of the video data after the incision is completed.

The object and advantages of the embodiments will be realized and achieved at least by the elements, features, and combinations particularly pointed out in the claims. It is to be understood that both the foregoing general description and the following detailed description are explanatory and are not restrictive of the invention, as claimed.

### BRIEF DESCRIPTION OF THE DRAWINGS

Example embodiments will be described and explained with additional specificity and detail through the use of the accompanying drawings in which:

FIG. 1A illustrates an example process flow to track incisions during a medical procedure;

FIG. 1B illustrates an example environment for tracking incisions during a medical procedure;

FIG. 2 illustrates another example process flow to track incisions during a medical procedure;

FIG. 3 illustrates a flowchart of an example method of tracking incisions;

FIG. 4 is a block diagram of an example computing system for generative artificial intelligence (AI); and

FIG. 5 is a block diagram of an example computing device, all in accordance with at least some embodiments of the present disclosure.

### DETAILED DESCRIPTION

During surgical procedures, surgeons may create multiple incisions in tissue to provide access points for surgical instruments. Due to the nature of some tissue, these incisions may become difficult to visualize through a surgical microscope once the incision-creating instrument has been withdrawn. For example, during ophthalmic surgery due to the clear and transparent color of the cornea, identifying the location of the incision through a microscope becomes a challenge.

Surgeons may rely on memory and trial-and-error approaches to relocate these incisions when inserting subsequent instruments, which may lead to inefficiency, potential tissue trauma from repeated probing, and surgical delays. Additionally, incision properties such as length, location, and angle may be estimated rather than precisely measured. However, these parameters may be used for other procedures or selection of medical devices. For example, the parameters may be used for toric intraocular lens power selection. As another example, the parameters may be used for residual astigmatism prediction in cataract surgery.

While some systems may automatically track incisions there may be high variability in instruments and timing in which incisions are made, making these systems unreliable. Additionally, variability in patient eyes may make it further challenging for a system to automatically detect an incision location and incision properties.

The embodiments described herein may address these challenges by providing an artificial intelligence-based system that may address one or more of these shortcomings. For example embodiments described herein may provide an artificial intelligence-based system that may automatically detect when an incision process begins during surgery, locate and track the incision in real-time as the incision is being created, and extract quantitative properties of the incision. Alternately or additionally, the system may register the incision location to a reference frame to allow the system to continuously overlay the incision location on live surgical video throughout a procedure regardless of changes in microscope settings or eye position. The system may thereby enable surgeons to maintain accurate spatial awareness of incision locations throughout a surgical procedure, may reduce time spent relocating incisions, may minimize unnecessary tissue manipulation, and may provide accurate measurements for surgical planning and intraocular lens calculations.

In some embodiments, the system may employ multiple specialized artificial intelligence modules that work together. For example, the system may include modules to detect incision initiation, track incision progression, detect incision completion, extract incision properties, and register and persist incision location across view transformations.

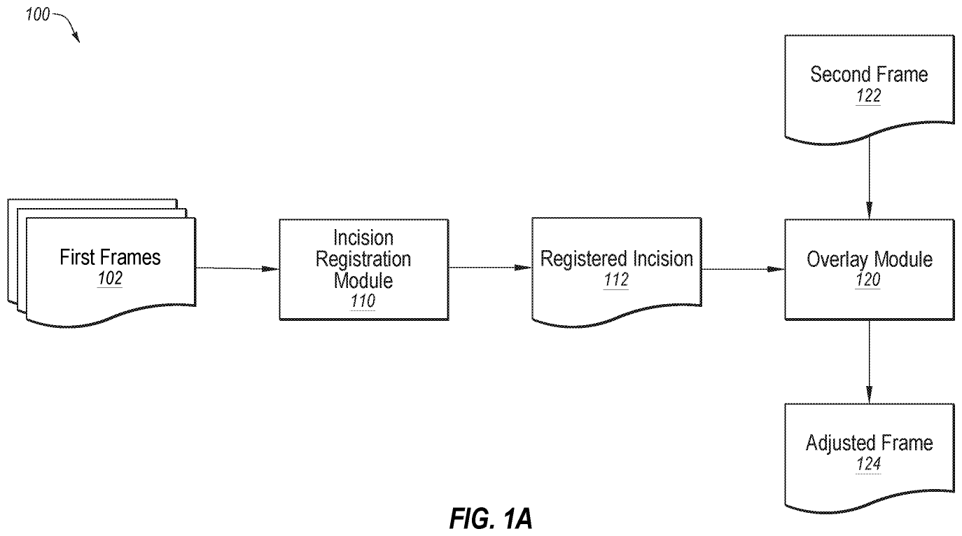

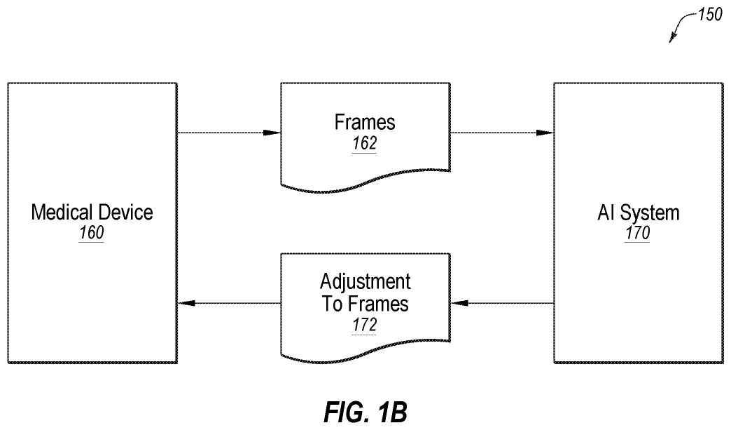

Turning to the figures, FIG. 1A illustrates an example process flow 100 to track incisions during a medical procedure. The process flow 100 may be arranged in accordance with at least one embodiment described in the present disclosure. The process flow 100 may include an incision registration module 110 and an overlay module 120.

In some embodiments, any operation performed by a module in this disclosure may be implemented in software, firmware, or a combination thereof. When implemented in software, the module may comprise one or more executable instructions stored on a non-transitory computer-readable medium and configured, when executed by one or more processors, to perform the operations described herein for that module. In this manner, the functionality attributed to the module may be carried out entirely through software execution, hardware execution, or any suitable combination of the two.

In some embodiments, the incision registration module 110 may obtain first frames 102. The first frames 102 may include or be any collection of frames captured during a surgical procedure. For example, the first frames 102 may include video frames captured by a surgical microscope camera during an ophthalmic surgical procedure. In these and other embodiments, the first frames 102 may be captured at a frame rate suitable for real-time processing, such as thirty frames per second, sixty frames per second, or any other suitable frame rate.

In some embodiments, the first frames 102 may include frames captured before, during, and after an incision creation process. The first frames 102 may include frames showing a medical tool making contact with tissue, frames showing the medical tool advancing through tissue to create an incision, and frames showing the medical tool being withdrawn after the incision is complete.

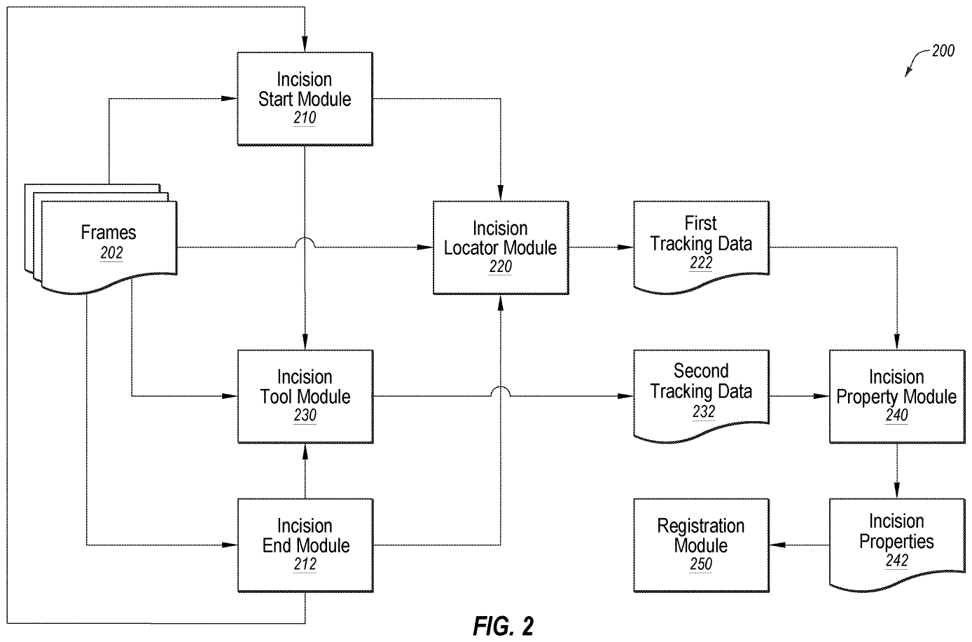

In some embodiments, the incision registration module 110 may be configured to analyze the first frames 102 to identify and register an incision location. The incision registration module 110 may receive the first frames 102 as input and may process the first frames 102 to detect when an incision process begins, track the incision as the incision is being created, and detect when the incision process ends. Based on the processing of the first frames 102, location coordinates of the incision may be registered. The incision registration module 110 may employ one or more artificial intelligence models to perform these operations, as described in greater detail below with respect to FIG. 2.

In some embodiments, the incision registration module 110 may select a representative frame from the first frames 102. The representative frame may be a frame that provides a view of the completed incision. For example, the representative frame may be a frame captured near the end or at the end of the incision creation process. In these and other embodiments, the incision registration module 110 may extract reference points from the representative frame. The reference points may be distinctive features in the representative frame that may be reliably detected and tracked across multiple frames.

In some embodiments, the incision registration module 110 may store the incision location coordinates in a representative frame coordinate system along with the extracted reference points. The incision location coordinates may be stored in a representative frame coordinate system along with the extracted reference points to enable persistent tracking of the incision location across subsequent frames regardless of view changes. By referencing the incision coordinates to the same coordinate system as the reference points, transformation parameters may be computed that may map the reference points from the representative frame to the reference points in any current frame. The same transformation parameters may be applied to the incision coordinates to thereby determine where the incision appears in a current frame, regardless of the view or zoom of the current frame being different than the representative frame. Thus, the representative frame coordinate system may provide a stable spatial reference frame that remains consistent even as the surgical field view changes due to zoom adjustments, lateral shifts, rotations, or other movements.

In some embodiments, besides the location coordinates of the incision, the incision registration module 110 may obtain various other properties of the incision. For example, the incision registration module 110 may obtain geometric measurements such as the length of the incision measured in pixels or converted to physical units such as millimeters using calibration data. As another example, the properties may also include angular measurements describing the orientation of the incision relative to a reference axis in the frame. In some embodiments, the properties may further include depth information indicating the penetration depth of the surgical tool into the tissue, a path descriptor characterizing the trajectory of the incision, such as whether the incision follows a straight line, a curved path, or a more complex pattern, an angle of the incision representing an angle of the medical tool when creating the incision,

In some embodiments, the incision registration module 110 may generate a registered incision 112 data structure. The registered incision 112 data structure may include or be data representing a location and properties of an incision that has been identified and registered by the incision registration module 110. For example, the registered incision 112 data structure may include coordinates defining the start point and end point of the incision in the representative frame coordinate system along with other properties of the incision. The registered incision 112 data structure may also include the reference points extracted from the representative frame. The registered incision 112 data structure may be provided to the overlay module 120.

In some embodiments, the overlay module 120 may obtain the registered incision 112 data structure. In these and other embodiments, the overlay module 120 may be configured to generate an overlay for a current frame indicating a location of an incision represented in the registered incision 112 data structure in the current frame. In these and other embodiments, to generate the overlay, the overlay module 120 may receive the registered incision 112 data structure and the second frame 122 as inputs. The overlay module 120 may determine a transformation between the representative frame and the second frame 122. The overlay module 120 may apply the transformation to the incision coordinates from the registered incision 112 data structure to determine where the incision appears in the second frame 122. The overlay module 120 may generate a graphical overlay or augmentation at the transformed incision location and may composite the overlay onto the second frame 122 to produce the adjusted frame 124. In some embodiments, the overlay may include a line or curve connecting the transformed start point and end point of the incision. The overlay may be rendered in a distinctive color such as yellow, cyan, red, or any other color that provides good visibility against the surgical field. In some embodiments, the overlay may have a transparency or opacity level that allows the underlying tissue to remain visible while making the overlay visible.

In some embodiments, the second frame 122 may include or be any frame captured during the surgical procedure after the incision has been created. In some embodiments, the second frame 122 may be captured at a time when a surgical view has changed from the view in the representative frame. For example, the surgical view may have been zoomed in or out, panned laterally, or rotated such that the second frame 122 may show the surgical field at a different magnification, position, or orientation than the representative frame.

In some embodiments, to determine the transformation between the representative frame and the second frame 122, the overlay module 120 may extract reference points from the second frame 122 using the technique used to identify the reference points from the representative frame. The overlay module 120 may match the reference points between the representative frame and the second frame 122. In these and other embodiments, the overlay module 120 may compute a transformation matrix that maps the reference points from the representative frame to the reference points in the second frame 122. The overlay module 120 may apply the transformation matrix to the incision coordinates from the registered incision 112 to determine the incision location in the second frame 122.

In some embodiments, the adjusted frame 124 may include or be the second frame 122 with the overlay or augmentation indicating the incision. The adjusted frame 124 may be displayed to a surgeon on a surgical monitor in real-time. The adjusted frame 124 may provide the surgeon with a visual indication of where the incision is located. Thus, the adjusted frame 124 may enable the surgeon to more quickly and accurately locate the incision for inserting subsequent surgical procedures.

Modifications, additions, or omissions may be made to the process flow 100 without departing from the scope of the present disclosure. For example, in some embodiments, the process flow 100 may process multiple incisions simultaneously. The incision registration module 110 may detect and register multiple incisions created at different times during the surgical procedure. The overlay module 120 may generate overlays for all registered incisions and may composite all overlays onto the second frame 122. In these and other embodiments, each incision overlay may be rendered in a different color or with a different visual style to distinguish between incisions.

As another example, the overlay module 120 may include additional information in the overlay beyond just the incision location. The overlay may include text annotations indicating the incision length, angle, or other properties. The overlay may include endpoint markers such as circles or crosses at the start and end points of the incision. The overlay may include a label such as “Main Incision” or “Paracentesis” to identify the type of incision.

As another example, the process module 100 may operate continuously throughout the surgical procedure. The overlay module 120 may thus receive a continuous stream of frames that includes the second frame 122 and may generate a continuous stream of adjusted frames 124. The adjusted frames 124 may be displayed in real-time on the surgical monitor, providing the surgeon with persistent visual guidance.

FIG. 1B illustrates an example environment 150 for tracking incisions during a medical procedure. The environment 150 may be arranged in accordance with at least one embodiment described in the present disclosure. The environment 150 may include a medical device 160, frames 162, an AI system 170, and adjusted frames 172.

In some embodiments, the medical device 160 may include or be any device used in a medical or surgical setting that includes imaging capabilities. For example, the medical device 160 may include a surgical microscope with an integrated digital camera. The medical device 160 may include an endoscope, a laparoscope, an ophthalmoscope, or any other medical imaging device. The medical device 160 may be configured to capture frames or video of a surgical field, a patient's anatomy, or any other subject relevant to a medical procedure.

In some embodiments, the medical device 160 may be configured to transmit the frames 162 to the AI system 170. The frames 162 may be transmitted in real-time as the frames 162 are captured. In some embodiments, the AI system 170 may include or be any computing system configured to perform artificial intelligence-based analysis of medical frames. For example, the AI system 170 may include a workstation computer with a graphics processing unit configured to execute neural network inference operations. Alternately or additionally, the AI system 170 may include a server computer or edge device located in an equipment room adjacent to an operating room. Alternately or additionally, the AI system 170 may include a cloud-based computing system accessible over a network and/or an embedded computing module integrated into the medical device 160.

In some embodiments, the AI system 170 may be configured to receive the frames 162 from the medical device 160 and to process the frames 162 to detect and track surgical incisions. In some embodiments, the AI system 170 may include the incision registration module 110 and the overlay module 120 described with respect to FIG. 1A. In these and other embodiments, the AI system 170 may perform the operations described with respect to the incision registration module 110 and/or the overlay module 120 to generate the adjustments to frames 172.

In some embodiments, the AI system 170 may be configured to transmit the adjustments to frames 172 back to the medical device 160. In some embodiments, the adjustments to frames 172 may include the frames 162 with incision overlays composited onto the frames 162. Alternately or additionally, the adjustments to frames 172 may include overlay or augmentation data to allow the medical device 160 to augment frames at the medical device 160. In some embodiments, the adjustments to frames 172 may be transmitted in real-time as the adjustments to frames 172 are generated.

In some embodiments, the medical device 160 may be configured to display frames with the adjustments to frames 172 to a user such as a surgeon. The medical device 160 may include a display screen integrated into the medical device 160. Alternately or additionally, the medical device 160 may be connected to an external display monitor. The display may provide the surgeon with a view of the surgical field with incision overlays indicating the locations of incisions.

Modifications, additions, or omissions may be made to the environment 150 without departing from the scope of the present disclosure. For example, in some embodiments, the AI system 170 may be integrated into the medical device 160 rather than being a separate system. In these embodiments, the frames 162 may not need to be transmitted to a separate AI system 170, and the adjustments to frames 172 may be generated internally within the medical device 160.

As another example, the AI system 170 may transmit additional data to the medical device 160 beyond the adjustments to frames 172. The AI system 170 may transmit incision property data such as incision lengths, angles, and locations. The medical device 160 may display this data to the surgeon in a text or graphical format. In these and other embodiments, the medical device 160 may store this data for post-operative review or for integration with electronic health record systems.

As another example, the environment 150 may include multiple medical devices 160 connected to the AI system 170. The AI system 170 may receive frames from the multiple medical devices 160 and may process frames from multiple medical devices 160 in overlapping time steps. The AI system 170 may transmit corresponding adjusted frames back to each medical device 160.

As another example, the AI system 170 may transmit the incision properties to the medical device 160 instead of the adjustments to the frames 172. In these and other embodiments, the medical device 160 may perform the operations of the overlay module 120 and generate the adjustments to the frames 172.

FIG. 2 illustrates another example process flow 200 to track incisions during a medical procedure. The process flow 200 may be arranged in accordance with at least one embodiment described in the present disclosure. The process flow 200 may include an incision start module 210, an incision end module 212, an incision locator module 220, an incision tool module 230, an incision property module 240, and a registration module 250.

In some embodiments, the process flow 200 may include frames 202. The frames 202 may include or be any collection of frame frames captured during a surgical procedure. The frames 202 may be similar to the first frames 102 described with respect to FIG. 1A. The frames 202 may be provided as input to multiple modules within the process flow 200, including the incision start module 210, the incision end module 212, and the incision tool module 230.

In some embodiments, the incision start module 210 may be configured to detect when an incision process begins. The incision start module 210 may receive the frames 202 as input and may analyze the frames 202 to identify frames where an incision initiation occurs. The incision start module 210 may employ an AI model to perform the detection. For example, the AI model may be an AI classifier model that is trained on annotated data of incision initiation.

In some embodiments, the incision start module 210 may analyze a current frame in the context of previous frames. The incision start module 210 may compare the current frame to one or more previous frames to detect changes that indicate an incision is beginning. For example, the incision start module 210 may detect the appearance of a surgical tool in contact with tissue, the appearance of a separation or opening in tissue, or changes in pixel intensity or color that indicate tissue disruption.

In some embodiments, the incision start module 210 may output a detection result indicating whether an incision has been detected in the current frame. Alternately or additionally, the detection result may include coordinates defining a bounding box around the detected incision region. In response to the incision start module 210 detecting an incision initiation, the incision start module 210 may activate the incision locator module 220 and the incision tool module 230. Alternately or additionally, in response to detecting an incision initiation, the incision start module 210 may activate the incision end module 212. In these and other embodiments, the incision start module 210 may cease operation until the incision end module 212 sends a signal that the process of making the incision has ended.

In some embodiments, the AI classifier model of the incision start module 210 may be trained on annotated surgical video data in which expert annotators have labeled frames corresponding to incision initiation events.

In some embodiments, the training dataset may include tens, hundreds, thousands, or more surgical video frames from multiple procedures, with each frame labeled to indicate whether an incision process has started at that point in time. The annotated data may capture variations across different surgical instruments, different patient anatomies, different lighting conditions, and different microscope settings to enable the model to generalize across diverse surgical scenarios. During training, the AI classifier model may learn to identify visual patterns and temporal changes in the video frames that are characteristic of incision initiation, such as tissue deformation, instrument contact with tissue, and changes in pixel intensity at the incision site. The training process may involve feeding sequences of frames to the AI classifier model, computing a loss function that measures the difference between the AI classifier model's predictions and the ground truth labels, and iteratively adjusting the AI classifier model's parameters through backpropagation to minimize this loss. The trained AI classifier model may then be validated on a separate set of annotated surgical videos to assess its accuracy in detecting incision starts before deployment in the clinical system.

In some embodiments, the incision locator module 220 may be configured to locate the precise position of the incision in the frames 202. The incision locator module 220 may be activated by the incision start module 210 when an incision initiation is detected. The incision locator module 220 may receive frames from the frames 202 and may process the frames to identify the incision location. The incision locator module 220 may employ an artificial intelligence model to perform segmentation of the incision region.

In some embodiments, the incision locator module 220 may generate a segmentation mask indicating which pixels in the frame belong to the incision. The segmentation mask may be a binary mask where pixels belonging to the incision have a value of one and pixels not belonging to the incision have a value of zero. Alternately or additionally, the segmentation mask may be a probability map where each pixel has a value between zero and one indicating the probability that the pixel belongs to the incision. The incision locator module 220 may extract the boundary coordinates of the incision from the segmentation mask.

In some embodiments, the incision locator module 220 may generate first tracking data 222. The first tracking data 222 may include coordinates defining the location of the incision in each frame. For example, the first tracking data 222 may include a sequence of incision boundary coordinates over time as the incision is being created. In some embodiments, the first tracking data 222 may include start point coordinates and end point coordinates for the incision. The first tracking data 222 may be provided as input to the incision property module 240.

In some embodiments, the AI model of the incision locator module 220 may be trained on annotated surgical video data in a similar manner as the AI model of the incision start module 210 is trained. In some embodiments, the incision locator module 220 may be deactivated by the incision end module 212 when the incision process is determined to be complete.

In some embodiments, the incision tool module 230 may be configured to detect and track the medical tool that is creating the incision. The incision tool module 230 may be activated by the incision start module 210 when an incision initiation is detected. The incision tool module 230 may receive frames from the frames 202 and may process the frames to identify the tool position. The incision tool module 230 may employ an AI model to perform object detection or segmentation of the medical tool.

In some embodiments, the incision tool module 230 may track the medical tool position over time as the incision is being created. In some embodiments, the incision tool module 230 may identify the tool tip position in each frame and/or the incision tool module 230 may track the tool trajectory as the medical tool moves through the tissue. In these and other embodiments, the incision tool module 230 may estimate the tool penetration depth based on the proportion of the medical tool visible in the frame or based on other visual cues. Alternately or additionally, the incision tool module 230 may also determine the medical tool orientation or angle relative to the tissue surface.

In some embodiments, the incision tool module 230 may generate second tracking data 232. The second tracking data 232 may include coordinates defining the position of the tool tip in each frame and a sequence of tool positions over time. The second tracking data 232 may also include tool orientation or angle information and tool depth information, among other information. The second tracking data 232 may be provided as input to the incision property module 240.

In some embodiments, the AI model of the incision tool module 230 may be trained on annotated surgical video data in a similar manner as the AI model of the incision start module 210 is trained. In some embodiments, the incision tool module 230 may be deactivated by the incision end module 212 when the incision process is determined to be complete.

In some embodiments, the incision end module 212 may be configured to detect when the incision process is complete. The incision end module 212 may receive the frames 202 as input and may analyze the frames 202 to identify when the incision creation has ended. The incision end module 212 may employ an artificial intelligence model with adaptive learning capabilities to perform this detection.

In some embodiments, the incision end module 212 may monitor the incision over time to determine when the process of creating the incision ends. For example, to determine that the process of creating the incision ends, the incision end module 212 may detect when the incision width begins to decrease, which may indicate that the medical tool is being withdrawn. Alternately or additionally, the incision end module 212 may detect when the incision length stabilizes, which may indicate that the incision has reached its final size. Alternately or additionally, the incision end module 212 may analyze the rate of change of incision length to determine when the incision process has ended. In some embodiments, the incision end module 212 may record the frame number and timestamp of the incision completion.

In some embodiments, when the incision end module 212 detects that the incision process is complete, the incision end module 212 may send a signal to deactivate the incision locator module 220 and the incision tool module 230. In these and other embodiments, the incision end module 212 may also send a signal to the incision start module 210 to begin to monitor for another incision. In some embodiments, the incision end module 212 may also send a signal to the incision property module 240 to finalize the incision property calculations.

In some embodiments, the AI model of the incision end module 212 may be trained on annotated surgical video data in a similar manner as the AI model of the incision start module 210 is trained.

In some embodiments, the incision property module 240 may be configured to calculate properties of the incision based on the first tracking data 222 and the second tracking data 232. The incision property module 240 may receive the first tracking data 222 from the incision locator module 220 and the second tracking data 232 from the incision tool module 230. The incision property module 240 may perform computational operations to extract quantitative properties of the incision.

In some embodiments, the incision property module 240 may calculate the incision length. The incision length may be calculated as the Euclidean distance between the start point and end point of the incision. Alternately or additionally, the incision length may be calculated as the arc length along the incision path if the incision follows a curved trajectory. The incision property module 240 may convert the length from pixels to physical units such as millimeters using a calibration factor that relates pixel distance to physical distance at the current microscope magnification.

In some embodiments, the incision property module 240 may calculate the incision angle. The incision angle may be calculated as the angle of the line connecting the start point and end point of the incision relative to a reference axis such as the horizontal axis of the frame. The incision angle may be expressed in degrees or radians. The incision angle may indicate the orientation of the incision on the tissue surface.

In some embodiments, the incision property module 240 may estimate the tool tip depth. The tool tip depth may indicate how far the medical tool penetrated into the tissue. The tool tip depth may be estimated based on visual cues in the frames such as the proportion of the medical tool that is visible or the appearance of tissue deformation around the medical tool. Alternately or additionally, if depth sensing hardware is available, the tool tip depth may be measured directly.

In some embodiments, the incision property module 240 may calculate the incision duration. The incision duration may be the time elapsed between the incision start frame and the incision end frame. The incision duration may be expressed in seconds or in number of frames. The incision duration may provide information about the speed at which the incision was created.

In some embodiments, the incision property module 240 may classify the incision path type. The incision path type may indicate whether the incision follows a straight path, a curved path, or a zigzag path. The incision property module 240 may analyze the sequence of medical tool positions or incision boundary coordinates to determine the path type. In some embodiments, the path type classification may be useful for characterizing different surgical techniques. In some embodiments, the incision property module 240 may select a representative frame from the incision creation sequence.

In some embodiments, the incision property module 240 may generate the incision properties 242. The incision properties 242 may include one or more of the calculated properties of the incision such as start point coordinates, end point coordinates, length, angle, depth, duration, and path type. The incision properties 242 may also include the identifier of the representative frame. The incision properties 242 may be provided as input to the registration module 250.

In some embodiments, the registration module 250 may receive the incision properties 242 from the incision property module 240. In these and other embodiments, the registration module 250 may be configured to register the incision location to enable persistent tracking across view transformations using the incision properties 242.

To register the incision location, the registration module 250 may extract reference points from the representative frame. The reference points may be distinctive features in the frame that may be reliably detected and tracked across other frames.

In some embodiments, the registration module 250 may use a reference point detection algorithm such as SIFT, SURF, ORB, or a learned feature detector to extract the reference points. The reference point detection algorithm may identify corners, edges, or other distinctive features in the representative frame. For each reference point, the algorithm may compute a descriptor that characterizes the local frame patch around the reference point. The descriptor may be a vector of numerical values that captures the appearance of the frame region.

In some embodiments, the registration module 250 may use the reference points to generate a representative frame coordinate system. In these and other embodiments, the registration module 250 may store the incision coordinates from the incision properties 242 in the representative frame coordinate system. Alternately or additionally, the registration module 250 may store the reference points and their descriptors. The stored information may be part of the registered incision data that may be used for generating overlays on subsequent frames. For example, as explained previously, for subsequent frames in the video after the incision is complete, reference points from the current frame may be extracted using the same reference points detection algorithm. The reference points between the representative frame and the current frame may be matched. A transformation matrix that maps coordinates from the representative frame to the current frame may be computed and may be applied to the incision coordinates from the incision properties 242 to determine where the incision appears in the current frame.

Modifications, additions, or omissions may be made to the process flow 200 without departing from the scope of the present disclosure. For example, in some embodiments, the incision start module 210, the incision locator module 220, the incision tool module 230, and the incision end module 212 may be implemented as separate artificial intelligence models that operate in parallel or in sequence. Alternately or additionally, some or all of these modules may be combined into a single unified artificial intelligence model that performs all detection, localization, and tracking operations.

As another example, the process flow 200 may process multiple incisions simultaneously. The incision start module 210 may detect multiple incision initiations at different times during the surgical procedure. The incision locator module 220 and the incision tool module 230 may track multiple incisions concurrently. The incision property module 240 may calculate properties for each incision separately. The registration module 250 may register multiple incisions and may generate overlays for all registered incisions on subsequent frames.

As another example, the incision property module 240 may calculate additional properties beyond those described above. The incision property module 240 may calculate the incision width or cross-sectional area. The incision property module 240 may estimate the quality of the incision based on the smoothness of the incision edges or other visual characteristics.

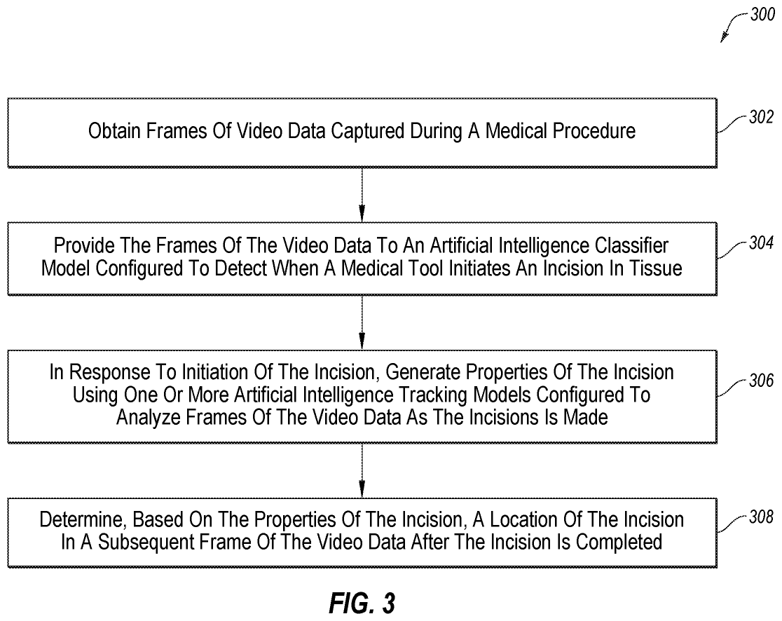

FIG. 3 illustrates a flowchart of an example method 300 for tracking an incision. The method 300 may be arranged in accordance with at least one embodiment described in the present disclosure. One or more operations of the method 300 may be performed, in some embodiments, by a device or system, such as the AI system 170 of FIG. 1B or the process flow 200 of FIG. 2, or another device or combination of devices. In these and other embodiments, the method 300 may be performed based on the execution of instructions stored on one or more non-transitory computer-readable media. Although illustrated as discrete blocks, various blocks may be divided into additional blocks, combined into fewer blocks, or eliminated, depending on the desired implementation.

The method 300 may begin at block 302, where frames of video data captured during a medical procedure may be obtained. The frames of video data may be captured by a surgical microscope camera or any other medical imaging device. The frames may be obtained in real-time as the frames are captured during the medical procedure.

At block 304, the frames of the video data may be provided to an artificial intelligence classifier model configured to detect when a medical tool initiates an incision in tissue. The artificial intelligence classifier model may be similar to the incision start module 210 described with respect to FIG. 2. The artificial intelligence classifier model may analyze each frame or a sequence of frames to detect changes indicative of incision initiation.

In some embodiments, the artificial intelligence classifier model may output a detection result for each frame indicating whether an incision initiation has been detected. In some embodiments, when the artificial intelligence classifier model detects an incision initiation, the method 300 may proceed to block 306.

At block 306, in response to initiation of the incision, properties of the incision may be generated using one or more artificial intelligence tracking models configured to analyze frames of the video data as the incision is made. In some embodiments, the one or more artificial intelligence tracking models may be similar to the incision locator module 220 and the incision tool module 230 described with respect to FIG. 2. In some embodiments, the one or more artificial intelligence tracking models may continue to analyze frames and generate properties as long as the incision is being created.

In some embodiments, the one or more artificial intelligence tracking models may include a first artificial intelligence tracking model configured to locate the incision in each frame. The first artificial intelligence tracking model may track the incision location over time as the incision is being created. In some embodiments, the one or more artificial intelligence tracking models may include a second artificial intelligence tracking model configured to track the surgical tool that is creating the incision.

In some embodiments, the properties of the incision may be calculated based on the outputs of the one or more artificial intelligence tracking models. The properties may include the incision start point coordinates, the incision end point coordinates, the incision length, the incision angle, the tool tip depth, the incision duration, and/or the incision path type, among others.

At block 308, based on the properties of the incision, a location of the incision in a subsequent frame of the video data after the incision is completed may be determined. In these and other embodiments, a representative frame may be selected from the frames captured during the incision creation process. The representative frame may be a frame that provides a clear view of the completed incision. Reference points may be extracted from the representative frame. The incision location coordinates in the representative frame may be stored along with the reference points.

In some embodiments, for a subsequent frame after the incision is completed, reference points may be extracted from the subsequent frame. The reference points from the subsequent frame may be matched to the reference points from the representative frame. A transformation matrix may be computed based on the matched reference points. The transformation matrix may map coordinates from the representative frame to the subsequent frame. The transformation matrix may be applied to the incision location coordinates from the representative frame to determine the incision location in the subsequent frame.

In some embodiments, an overlay may be generated at the determined incision location in the subsequent frame. The subsequent frame with the overlay may be displayed to a user such as a surgeon, providing a visual indication of where the incision is located despite changes in the microscope view.

It is understood that, for this and other processes, operations, and methods disclosed herein, the functions and/or operations performed may be implemented in differing order. Furthermore, the outlined functions and operations are only provided as examples, and some of the functions and operations may be optional, combined into fewer functions and operations, or expanded into additional functions and operations without detracting from the essence of the disclosed embodiments.

For example, the method 300 may include detecting when the incision is completed. A second artificial intelligence classifier model may be configured to detect when the incision process ends. The second artificial intelligence classifier model may monitor the incision length over time and may detect when the incision length begins to decrease or stabilize, indicating that the tool is being withdrawn and the incision is complete. When the incision is detected as complete, the one or more artificial intelligence tracking models may be deactivated.

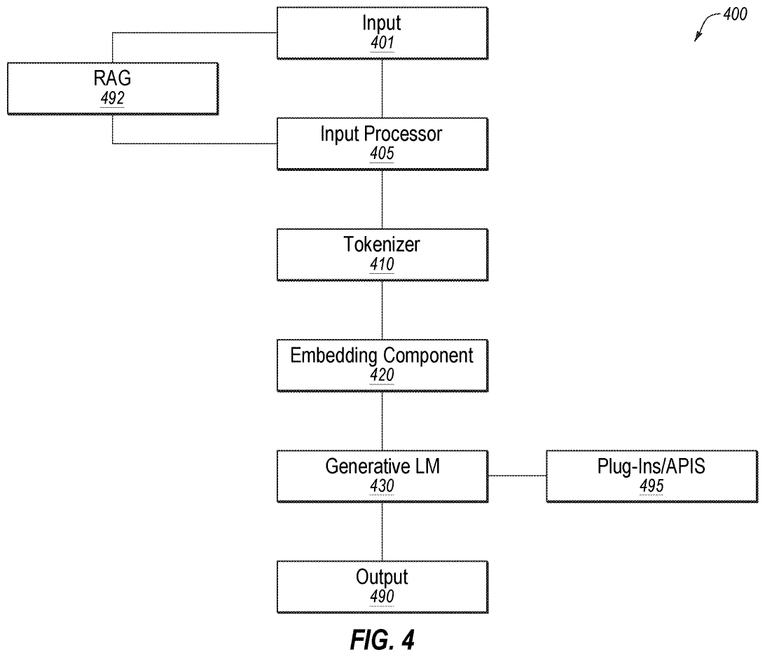

FIG. 4 is a block diagram of an example generative language model system 400 suitable for use in implementing at least some embodiments of the present disclosure. In the example illustrated in FIG. 4, the generative language model system 400 includes a retrieval augmented generation (RAG) component 492, an input processor 405, a tokenizer 410, an embedding component 420, plug-ins/APIs 495, and a generative language model (LM) 430 (which may include an LLM (large language model), a VLM (vision language model), a MMLM (multi-modal language model), a VLA (vision language action) model, etc.). The generative language model system 400 may be an example of a system that performs the AI models in the modules described with respect to FIG. 1A and FIG. 2.

At a high level, the input processor 405 may receive an input 401 comprising text and/or other types of input data (e.g., audio data, video data, frame data, sensor data), depending on the architecture of the generative LM 430 (e.g., LLM/LRM/VLM/MMLM/etc.). Taking raw input text as an example, the input processor 405 may prepare raw input text in various ways. For example, the input processor 405 may perform various types of text filtering to remove noise (e.g., special characters, punctuation, HTML (Hypertext Markup Language) tags, stopwords, portions of an frame(s), portions of audio, etc.) from relevant textual content.

In some embodiments, a RAG component 492 (which may include one or more RAG models, and/or may be performed using the generative LM 430 itself) may be used to retrieve additional information to be used as part of the input 401 or prompt. RAG may be used to enhance the input to the LLM/LRM/VLM/MMLM/etc. with external knowledge, so that answers to specific questions or queries or requests are more relevant-such as in a case where specific knowledge is required. The RAG component 492 may fetch this additional information (e.g., grounding information, such as grounding text/frame/video/audio/USD/CAD/etc.) from one or more external sources, which can then be fed to the LLM/LRM/VLM/MMLM/etc. along with the prompt to improve accuracy of the responses or outputs of the model.

For example, in some embodiments, the input 401 may be generated using the query or input to the model (e.g., a question, a request, etc.) in addition to data retrieved using the RAG component 492. In some embodiments, the input processor 405 may analyze the input 401 and communicate with the RAG component 492 (or the RAG component 492 may be part of the input processor 405, in embodiments) in order to identify relevant text and/or other data to provide to the generative LM 430. The RAG component 492 may use various RAG techniques such as, naïve RAG, modular RAG techniques, and/or Graph RAG.

The tokenizer 410 may segment the (e.g., processed) text data into smaller units (tokens) for subsequent analysis and processing. The tokens may represent individual words, subwords, characters, portions of audio/video/frame/etc., depending on the implementation. For example, the tokenizer 410 may convert the (e.g., processed) text into a structured format according to tokenization schema being implemented in the particular embodiment.

The embedding component 420 may use any known embedding technique to transform discrete tokens into (e.g., dense, continuous vector) representations of semantic meaning. For example, the embedding component 420 may use pre-trained word embeddings (e.g., Word2Vec, GloVe, or FastText), one-hot encoding, Term Frequency-Inverse Document Frequency (TF-IDF) encoding, one or more embedding layers of a neural network, and/or otherwise. In some implementations in which the input 401 includes video data, the input processor 401 may extract frames or apply resizing to extracted frames, and the embedding component 420 may extract features such as optical flow embeddings or video embeddings and/or may encode temporal information or sequences of frames.

The generative LM 430 and/or other components of the generative LM system 400 may use different types of neural network architectures depending on the implementation. For example, transformer-based architectures such as those used in models like GPT (Generative Pre-trained Transformer) may be implemented and may include self-attention mechanisms. Some other non-limiting example architectures include transformers (e.g., encoder-decoder, decoder only, multi-modal), RNNs (Recurrent Neural Network), LSTMs (Long Short-Term Memory), fusion models, diffusion models, cross-modal embedding models that learn joint embedding spaces, graph neural networks (GNNs), hybrid architectures combining different types of architectures adversarial networks like generative adversarial networks or GANs or adversarial autoencoders (AAEs) for joint distribution learning, linear-time sequence modeling with selective state space modeling (SSM) architectures (e.g., Mamba LLM architectures), and/or others. As such, depending on the implementation and architecture, the embedding component 420 may apply an encoded representation of the input 401 to the generative LM 430, and the generative LM 430 may process the encoded representation of the input 401 to generate an output 490, which may include responsive text and/or other types of data.

As described herein, in some embodiments, the generative LM 430 may be configured to access or use—or capable of accessing or using—plug-ins/APIs 495 (which may include one or more plug-ins, application programming interfaces (APIs), databases, data stores, repositories, etc.). These and other architectures for LLMs/VLMs/MMLMs/VLAs/etc. described herein are meant simply as examples, and other suitable architectures may be implemented within the scope of the present disclosure.

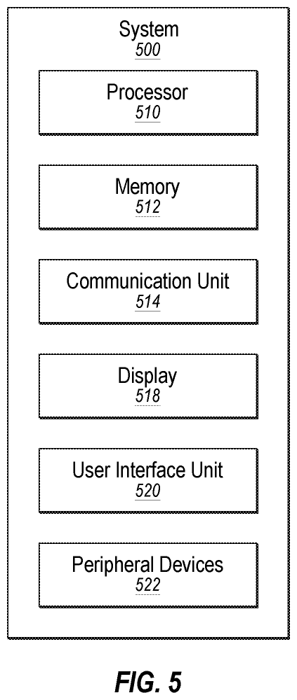

FIG. 5 illustrates an example system 500 that may be used during transcription of communications. The system 500 may be arranged in accordance with at least one embodiment described in the present disclosure. The system 500 may include a processor 510, memory 512, a communication unit 516, a display 518, a user interface unit 520, and a peripheral device 522, which all may be communicatively coupled. In some embodiments, the system 500 may be part of any of the systems or devices described in this disclosure. For example, the system 500 may be part of the AI system 170 or the medical device 160. Alternately or additionally, the system 500 may be used to perform one or more operations of the modules described with respect to FIGS. 1A and 2.

Generally, the processor 510 may include any suitable special-purpose or general-purpose computer, computing entity, or processing device including various computer hardware or software modules and may be configured to execute instructions stored on any applicable computer-readable storage media. For example, the processor 510 may include a microprocessor, a microcontroller, a parallel processor such as a graphics processing unit (GPU) or tensor processing unit (TPU), a digital signal processor (DSP), an application-specific integrated circuit (ASIC), a Field-Programmable Gate Array (FPGA), quantum computer, or any other digital or analog circuitry configured to interpret and/or to execute program instructions and/or to process data.

Although illustrated as a single processor in FIG. 5, it is understood that the processor 510 may include any number of processors distributed across any number of networks or physical locations that are configured to perform individually or collectively any number of operations described herein. In some embodiments, the processor 510 may interpret and/or execute program instructions and/or process data stored in the memory 512. In some embodiments, the processor 510 may execute the program instructions stored in the memory 512.

For example, in some embodiments, the processor 510 may execute program instructions stored in the memory 512 that are related to selecting a transcription generation technique for a communication session such that the system 500 may perform or direct the performance of the operations associated therewith as directed by the instructions. In these and other embodiments, the instructions may be used to perform one or more operations of the methods described in this disclosure.

The memory 512 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 510.

By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store particular program code in the form of computer-executable instructions or data structures and which may be accessed by a general-purpose or special-purpose computer. Combinations of the above may also be included within the scope of computer-readable storage media.

Computer-executable instructions may include, for example, instructions and data configured to cause the processor 510 to perform a certain operation or group of operations as described in this disclosure. In these and other embodiments, the term “non-transitory” as explained in the present disclosure should be construed to exclude only those types of transitory media that were found to fall outside the scope of patentable subject matter in the Federal Circuit decision of In re Nuijten, 500 F. 4d 1446 (Fed. Cir. 2007). Combinations of the above may also be included within the scope of computer-readable media.

The communication unit 516 may include any component, device, system, or combination thereof that is configured to transmit or receive information over a network. In some embodiments, the communication unit 516 may communicate with other devices at other locations, the same location, or even other components within the same system. For example, the communication unit 516 may include a modem, a network card (wireless or wired), an infrared communication device, a wireless communication device (such as an antenna), and/or chipset (such as a Bluetooth device, an 802.6 device (e.g., Metropolitan Area Network (MAN)), a WiFi device, a WiMax device, cellular communication facilities, etc.), and/or the like. The communication unit 516 may permit data to be exchanged with a network and/or any other devices or systems described in the present disclosure.

The display 518 may be configured as one or more displays, like an LCD (liquid crystal display), LED (light emitting diode), OLED (organic light emitting diode), Braille terminal, or other type of display. The display 518 may be configured to present video, text captions, user interfaces, and other data as directed by the processor 510. The user interface unit 520 may include any device to allow a user to interface with the system 500. For example, the user interface unit 520 may include a mouse, a track pad, a keyboard, buttons, camera, and/or a touchscreen, among other devices. The user interface unit 520 may receive input from a user and provide the input to the processor 510. In some embodiments, the user interface unit 520 and the display 518 may be combined.

The peripheral devices 522 may include one or more devices. For example, the peripheral devices may include a microphone, a framer, and/or a speaker, among other peripheral devices. Modifications, additions, or omissions may be made to the system 500 without departing from the scope of the present disclosure. For example, in some embodiments, the system 500 may include any number of other components that may not be explicitly illustrated or described. Further, depending on certain implementations, the system 500 may not include one or more of the components illustrated and described.

As indicated above, the embodiments described herein may include the use of a special purpose or general-purpose computer (e.g., the processor 510 of FIG. 5) including various computer hardware or software modules, as discussed in greater detail below. Further, as indicated above, embodiments described herein may be implemented using computer-readable media (e.g., the memory 512 of FIG. 5) for carrying or having computer-executable instructions or data structures stored thereon.

Generally speaking, network environments suitable for use in implementing embodiments of the disclosure may include one or more client devices, servers, network attached storage (NAS), other backend devices, and/or other device types. The client devices, servers, and/or other device types (e.g., each device) may be implemented on one or more instances of the computing systems(s) 500 of FIG. 5—e.g., each device may include similar components, features, and/or functionality of the computing systems(s) 500. In addition, where backend devices (e.g., servers, NAS, etc.) are implemented, the backend devices may be included as part of a data center (such as, but not limited to, those described herein).

Components of a network environment may communicate with each other via a network(s), which may be wired, wireless, or both. The network may include multiple networks, or a network of networks. Compatible network environments may include one or more peer-to-peer network environments—in which case a server may not be included in a network environment—and one or more client-server network environments—in which case one or more servers may be included in a network environment. In peer-to-peer network environments, functionality described herein with respect to a server(s) may be implemented on any number of client devices.

In at least one embodiment, a network environment may include one or more cloud-based network environments, a distributed computing environment, a combination thereof, etc. A cloud-based network environment may be private (e.g., limited to a single organization), may be public (e.g., available to many organizations), and/or a combination thereof (e.g., a hybrid cloud environment).

The various features illustrated in the drawings may be, but are not necessarily, drawn to scale. The illustrations presented in the present disclosure are not meant to be actual views of any particular apparatus (e.g., device, system, etc.) or method but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used in the present disclosure and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” among others).

Relative terms used in the present disclosure and especially in the appended claims (e.g., bodies of the appended claims) are generally intended as falling within manufacturing tolerances and/or within scope reasonably understood by a person of skill in the art. For example, if two components are identified as being the “same” size, there may be variations consistent with manufacturing variances. Terms describing “approximately,” “similar,” “substantially,” or other terms designating similarity may convey within ten percent of the comparative value. For example, two components that are approximately the same size would be understood to be of a size within ten percent of each other.

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absent a showing that the terms “first,” “second,” “third,” etc., connote a specific order, these terms should not be understood to connote a specific order. Furthermore, absent a showing that the terms “first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited in the present disclosure are intended for pedagogical objects to aid the reader in understanding the invention and the concepts contributed by the inventor to furthering the art and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

What is claimed is:

1. A method of tracking an incision, the method comprising:
obtaining frames of video data captured during a medical procedure;
providing the frames of the video data to an artificial-intelligence classifier model configured to detect when a medical tool initiates an incision in tissue;
in response to initiation of the incision, generating properties of the incision using one or more artificial-intelligence tracking models configured to analyze frames of the video data as the incision is made; and
determining, based on the properties of the incision, a location of the incision in a subsequent frame of the video data after the incision is completed.

2. The method of claim 1, further comprising:
in response to initiation of the incision, analyzing the frames of the video data by a second artificial-intelligence classifier model to determine when the incision is completed; and
in response to the second artificial-intelligence classifier model determining that the incision is completed, ceasing operation of the one or more artificial-intelligence tracking models such that the properties of the incision are no longer produced.

3. The method of claim 1, further comprising registering one or more reference points in a registration frame that includes the incision being made, wherein the location of the incision is determined based on the one or more reference points.

4. The method of claim 3, wherein the registration frame is a last frame of the incision being made.

5. The method of claim 3, wherein determining the location of the incision includes:
computing transformation parameters by mapping the one or more reference-points from the registration frame to the subsequent frame; and
calculating the location of the incision in the subsequent frame using the transformation parameters and the properties of the incision.

6. The method of claim 1, wherein the one or more artificial-intelligence tracking models include a model configured to track movements of the medical tool while the medical tool makes the incision.

7. The method of claim 1, wherein the one or more artificial-intelligence tracking models include a model configured to determine locations where the incision has occurred.

8. The method of claim 1, wherein the properties of the incision include one or more of: a length of the incision, a beginning location of the incision, an ending location of the incision, a shape of the incision, a medical tool tip depth location, a medical tool x-y plane rotation, and an angle of the incision.

9. A system to track an incision, the system comprising:
one or more computer-readable media configured to store instructions; and
one or more processors coupled to the one or more computer-readable media and configured to execute the instructions to cause the system to perform operations, the operations comprising:
obtaining frames of video data captured during a medical procedure;
providing the frames of the video data to an artificial-intelligence classifier model configured to detect when a medical tool initiates an incision in tissue;
in response to initiation of the incision, generating properties of the incision using one or more artificial-intelligence tracking models configured to analyze frames of the video data as the incision is made; and
determining, based on the properties of the incision, a location of the incision in a subsequent frame of the video data after the incision is completed.

10. The system of claim 9, wherein the operations further comprise:
in response to initiation of the incision, analyzing the frames of the video data by a second artificial-intelligence classifier model to determine when the incision is completed; and
in response to the second artificial-intelligence classifier model determining that the incision is completed, ceasing operation of the one or more artificial-intelligence tracking models such that the properties of the incision are no longer produced.

11. The system of claim 9, wherein the operations further comprise registering one or more reference points in a registration frame that includes the incision being made, wherein the location of the incision is determined based on the one or more reference points.

12. The system of claim 11, wherein the registration frame is a last frame of the incision being made.

13. The system of claim 11, wherein determining the location of the incision includes:
computing transformation parameters by mapping the one or more reference-points from the registration frame to the subsequent frame; and
calculating the location of the incision in the subsequent frame using the transformation parameters and the properties of the incision.

14. The system of claim 9, wherein the one or more artificial-intelligence tracking models include a model configured to track movements of the medical tool while the medical tool makes the incision.

15. The system of claim 9, wherein the one or more artificial-intelligence tracking models include a model configured to determine locations where the incision has occurred.
